(19) United States
(12) Patent Application Publication
(10) Pub. No.: US 2021/0395185 A1
(43) Pub. Date: Dec. 23, 2021

(54) SCALABLE SYNTHESIS OF OPTICALLY ACTIVE 1-CYCLOPROPYLALKYL-1-AMINES

(71) Applicant: Boehringer Ingelheim International GmbH, Ingelheim am Rhein (DE)

(72) Inventors: Da DENG, Shanghai (CN); Zhanquan DU, Shanghai (CN); Klaus GROLL, Ingelheim am Rhein (DE); Sabrina HOFEDITZ, Wackernheim (DE); Marion LAUBE, Niederheimbach (DE); Michael PANGERL, Waldalgesheim (DE); Christian RINK, Wiesbaden (DE); Qing XIAO, Shanghai (CN); Zhibin ZHU, Shanghai (CN)

(21) Appl. No.: 17/285,564

(22) PCT Filed: Oct. 17, 2019
(86) PCT No.: PCT/EP2019/078208

§ 371 Date: Apr. 15, 2021

(30) Foreign Application Priority Data
Oct. 18, 2018 (CN) PCT/CN2018/110852

(51) Int. Cl. C07C 209/28; B01J 23/44

(52) U.S. Cl. CPC C07C 209/28; B01J 23/44

(57) ABSTRACT

The present invention provides a new, scalable synthetic method for the preparation of non-racemic 1-cyclopropyl alkyl-1-amines, e.g. (S)-1-cyclopropyl ethyl-1-amine. The method makes use of inexpensive starting materials (such as cyclopropyl methyl ketone and S-(−)-α-phenylethylamine) and is well suited for a large scale, industrial process.

### TECHNICAL FIELD

This application relates to a method of synthesis of 1-cyclopropyl ethyl-1-amine which is a building block in the preparation of substituted pyrazinones. These substituted pyrazinones can be used to prepare pharmaceutically active compounds containing a substituted pyrazinone ring system.

### BACKGROUND OF THE INVENTION

Cyclopropyl alkyl amines may be prepared by methods known in the literature and converted to substituted pyrazinones by adapting methods known in the literature. These substituted pyrazinone compounds can then be used to prepare pharmaceutically active compounds, such as ROR gamma modulators, containing a pyrazinone ring. These ROR gamma modulators are useful in treating a variety of diseases and disorders that are mediated through this pathway. The diseases that may be treated include but are limited to psoriasis and other inflammatory diseases. The preparation of ROR gamma modulators, containing a substituted pyrazinone ring, is disclosed in U.S. Pat. No. 9,242,989, WO2017/058831 or WO2017/127375.

Various synthetic routes to prepare non-racemic 1-cyclopropyl ethyl-1-amine have been described in the literature:

Asymmetric Catalysis by Transition Metals:

Angew. Chem. Int. Ed. 2014, 53, 1399-1403 offers a route using an Ir-catalyst for the synthesis of p-methoxyphenyl (PMP) protected cyclopropyl ethylamine. The proposed route uses relatively high loads of the Ir-catalyst (5 mol %, and 10 mol % of a bis-naphthyl ligand)

Org. Lett. 2009, 11, 4204-4207 discloses a route to PMP protected cyclopropyl ethylamine starting from cyclopropyl alkyne using a (expensive) gold catalyst. Further removal of the PMP protecting group seems not unproblematic, and oxidative cleavage results in the formation of quinones.

Chiral Resolution from Cyclopropyl Precursors:

J. Med. Chem. 2011, 54, 7334-7349 and WO2009/075830 disclose a route starting from cyclopropylaldehyde and a chiral sulfinamide. Similarly, U.S. 62/482,250 discloses a reaction sequence providing (S)-1-cyclopropylethan-1-amin hydrochloride in 76% overall yield from cyclopropanecarbaldehyde. However, the use of a low temperature Grignard reaction and purification by flash chromatography are drawbacks in view of a production in larger scale.

### BRIEF SUMMARY OF THE INVENTION

The present invention provides a new route towards non-racemic 1-cyclopropyl alkyl-1-amines. The synthesis is scalable and makes use of inexpensive starting materials (such as cyclopropyl methyl ketone and S-(−)-α-phenylethylamine). The route according to the present invention is well suited for a large scale, industrial process to manufacture non-racemic 1-cyclopropyl ethyl-1-amine, e.g. (S)-1-cyclopropyl ethyl-1-amine.

### DETAILED DESCRIPTION OF THE INVENTION

In a first aspect the present invention provides a process for preparing non-racemic 1-cyclopropyl alkyl-1-amines of formula I, e.g. (S)-1-cyclopropyl alkyl-1-amines

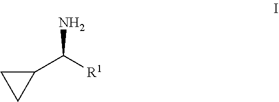

comprising the reaction of a compound of formula II

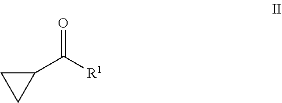

with a compound of formula III

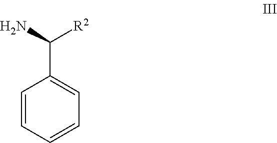

in which R¹ and R² are independently C₁-C₆-alkyl.

In a further aspect the process for preparing 1-cyclopropyl alkyl-1-amines of formula I comprises the steps of i) condensation of a compound of formula II with a compound of formula III to form an imine of formula INT1, ii) reduction to the corresponding secondary amine of formula INT2 and iii) debenzylation to the primary amine of formula I.

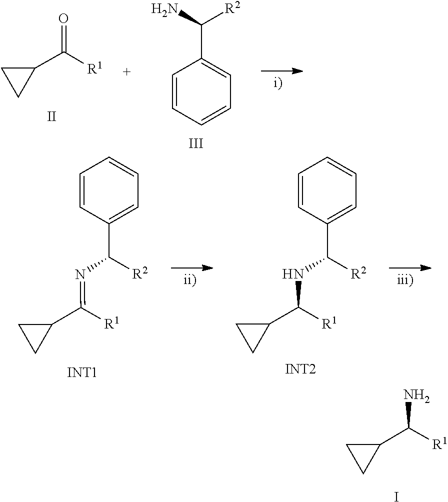

Preferred reaction conditions of step i) comprise the use of a Lewis acid in a suitable solvent.

Examples of solvents useful for reaction step i) include methanol, ethanol, iso-propanol, benzene, toluene, hexane, heptane, cyclopentane, cyclohexane, THF, 2-MeTHF, and isopropyl acetate or mixtures thereof. Preferred solvents are iso-propanol, Toluene, heptane, THF and 2-MeTHF or mixtures thereof. In a more specific aspect the solvent is is THF.

Examples of suitable Lewis acids include B(OiPr)₃ and Ti(OiPr)₄. In a more specific aspect the Lewis acid is Ti(OiPr)₄.

Preferred reaction conditions of step ii) comprise the use of NaBH₄ or LiBH₄ in a suitable solvent.

Examples of solvents useful for reaction step ii) include alcohols like methanol, ethanol, and iso-propanol, or THF or mixtures thereof. In a more specific aspect the solvent is ethanol, THF or mixtures thereof.

Preferred reaction conditions of step iii) comprise the use of Pd as catalyst under hydrogen atmosphere in a suitable solvent.

More specifically, the Pd catalyst is Pd on charcoal (Pd/C or Pd(OH)₂/C). Examples of solvents useful for reaction step iii) include alcohols like methanol, ethanol, and iso-propanol or mixtures thereof. In a more specific aspect the solvent is ethanol.

The optical purity of 1-cyclopropyl alkyl-1-amines of formula I obtained from the reaction of II with III is 60% ee or higher. In a further aspect the optical purity is 65% ee or higher. In a further aspect the optical purity is 70% ee or higher. In further aspects the optical purity is between 60% ee and 90% ee, between 60% ee and 80% ee, or between 65% ee and 75% ee, respectively.

In more specific embodiments R¹ is C₁₋₃-alkyl, e.g. R¹ is methyl, i.e the compound of formula II is cyclopropyl methyl ketone.

In a more specific embodiment R² is methyl, i.e. the compound of formula III is (S)-(−)-α-phenylethylamine.

In an additional aspect the compound of formula I is reacted with enantiomeric pure acids to form salts in order to further increase the isomeric purity of 1-cyclopropyl alkyl-1-amines. For example, the compounds of formula I can be converted into the corresponding mandelic acid salts. Therefore, in a further aspect the invention further comprises reacting the amine of formula I with mandelic acid. e.g. (R)-mandelic acid, in a suitable solvent, to provide a compound of formula IV:

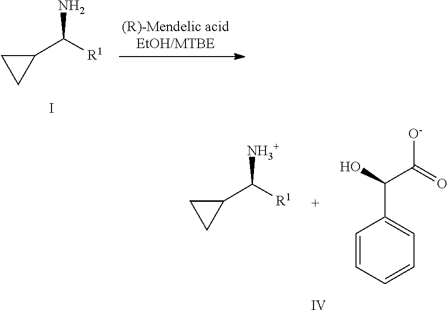

Examples of solvents include N,N-dimethylformamide, dichloromethane, ethyl acetate, hexane, heptane, acetonitrile, methyl tert-butyl ether (MTBE), isopropyl acetate, toluene, cyclopropylmethyl ether, and mixtures thereof. In a more specific aspect the solvent is ethanol, methyl tert-butyl ether or mixtures thereof.

The mandelate salt of formula IV can be converted back to the free base of formula I by treatment with bases like aq. NaOH.

The conversion to the mandelic acid salt and consequent crystallization increases the optical purity of 1-cyclopropyl alkyl-1-amines of formula I, e.g. of 1-cyclopropyl ethyl-1-amine.

The optical purity of 1-cyclopropyl alkyl-1-amines of formula I (e.g. of (S)-1-cyclopropyl ethyl-1-amine) obtained from the reaction of II with III is 97% ee or higher. In a further aspect the optical purity is 98% ee or higher. In a further aspect the optical purity is 99% ee or higher. In further aspects the optical purity is 99.5% ee or higher.

The conversion from compounds of formula II to compounds of formula I is suitable to be performed without the need of specific purification steps, e.g purification by chromatography.

Further, the conversion from compounds of formula II to compounds of formula IV is suitable to be performed without the need of specific purification steps, e.g purification by chromatography.

### USED TERMS AND DEFINITIONS

The term “C₁₋ₙ-alkyl”, wherein n is an integer selected from 2, 3, 4, 5 or 6, preferably 4 or 6, either alone or in combination with another radical denotes an acyclic, saturated, branched or linear hydrocarbon radical with 1 to n C atoms. For example the term C₁₋₅-alkyl embraces the radicals H₃C—, H₃C—CH₂—, H₃C—CH₂—CH₂—, H₃C—CH(CH₃)—, H₃C—CH₂—CH₂—CH₂—, H₃C—CH₂—CH(CH₃)—, H₃C—CH(CH₃)—CH₂—, H₃C—C(CH₃)₂—, H₃C—CH₂—CH₂—CH₂—CH₂—, H₃C—CH₂—CH₂—CH(CH₃)—, H₃C—CH₂—CH(CH₃)—CH₂—, H₃C—CH(CH₃)—CH₂—CH₂—, H₃C—CH₂—C(CH₃)₂—, H₃C—C(CH₃)₂—CH₂—₂—, H₃C—CH(CH₃)—CH(CH₃)— and H₃C—CH₂—CH(CH₂CH₃)—.

### Abbreviations

$$ee enantiomeric excess ;$$

$$\% ee = \frac{area \% of S isomer - area \% of R isomer}{area \% of S isomer + area \% of R - isomer}$$

GC gas chromatography
GC-MS coupled gas chromatography-mass spectrometry
IPA isopropanol
IPAc Isopropyl acetate
s MTBE methyl t-butyl ether
Pd/C Pd on charcoal
THF tetrahydrofuran
MeTHF 2-methyl tetrahydrofuran
Rt retention time (in GC/MS)

Methods:

GC Methods:

GC Method 1 (In-Process Control)

Instrument: GCMS Agilent 7890B GC system FID, 5977A MSD; column: DB-5 MS, L=30 m, ID=0.25 mm, Film=0.5 μm; Diluent: MTBE and 2M NaOH; Carrier gas: Helium (constant flow=1.2 mL/min); Injection mode: split 1:10; Injector Temp: 220° C.; FID Temp: 280° C.; Oven Temp gradient: 40° C. (5 min)→15° C./min→280° C. (2 min); Ion source: EI; scan range: 2˜550 amu

Method 2 (Purity Determination)

Instrument: GC Agilent 7890A GC system FID; column: CP-Volamine Agilent, L=30 m, ID=0.32 mm, Film=5 μm; Diluent: MTBE; Carrier gas: Helium (constant flow=2.5 mL/min); Injection mode: split 1:10; Injector Temp: 220° C.; FID Temp: 240° C.; Oven Temp gradient: 40° C. (5 min)→10° C./min→220° C. (2 min).

Method 3 (Chiral Determination)

Instrument: GC Agilent 7890B GC system FID; column: Supelco BetaDex 120, L=30 m, ID=0.25 mm, Film=0.25 μm; Diluent: DCM; Carrier gas: Helium (constant flow=2.0 mL/min); Injection mode: split 1:30; Injector Temp: 220° C.; FID Temp: 230° C.; Oven Temp gradient: 75° C. (18 min)→10° C./min→120° C. (2.5 min).

Method 4 (MS Determination)

Instrument: Agilent GC7890A\MS5975C system MS; column: Rxi 624 Sil MS, L=20 m, ID=0.18 mm, Film=1.0 μm; Carrier gas: Helium (constant flow=0.8 mL/min); Injection mode: split 1:30; detector temp: 325° C.; Inlet temp: 250° C.; Oven Temp gradient: 40° C. (0 min)→30° C./min→300° C. (3.3 min); MS scan parameters: EMV mode: relative; mass low: 30.0, high: 500.0; mass threshold: 100.0; MS source: 230° C.; MS Quad 150° C.

Preparation

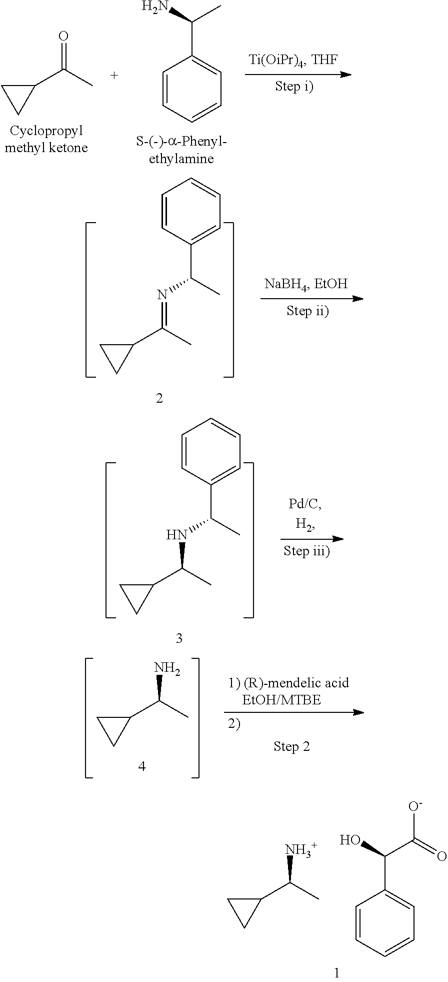

Steps i) to iii):

A mixture of (S)-(−)-α-phenylethylamine (100 g) and cyclopropyl methyl ketone (72.9 g) n THF (200 mL) was stirred at room temperature. Ti(OiPr)₄ (249 g) was added over 30 min. The mixture was heated to 70° C. and hold for 3 h, then cooled to 0° C. and NaBH₄ (18.8 g) was added. The suspension was stirred at 0° C. for 1 h, then EtOH (200 mL) was added slowly and stirred for 1 h. THF (500 mL) and celite (60 g) was added and the reaction was quenched with water (100 mL). The suspension was stirred at 25° C. for 1 h and 40 wt % aqueous NaOH (200 g) was added. The mixture was filtered and washed with THF (twice 200 mL). The filtrate was concentrated to remove solvents. Water (100 mL) was added, extracted with MTBE (500 mL) and washed with water (200 mL). The organic layer was concentrated to dryness and diluted with EtOH (600 mL). 10% Pd/C (9.3 g) was added to the solution, transferred to a 2 L hydrogenator, and stirred under H₂ (10 bar) at 70° C. for 24 h. The reaction mixture was cooled to 25° C. and was filtered through celite to remove catalyst. The filtrate was the solution of 4 in EtOH and it was used in the next step directly (assay by GC (Method 1): 59.7 g, yield 85%, calculated from (S)-(−)-α-phenylethylamine, e/r 84/16).

GC (Method 1): R₄=3.11 min

MS (Method 4): m/z=85.1 M⁺

Table 1 and Table 2 summarise further conditions of the condensation of cyclopropyl methyl ketone (“ketone” in Table 1) and (S)-(−)-1-phenylethylamine (“amine” in Table 1)

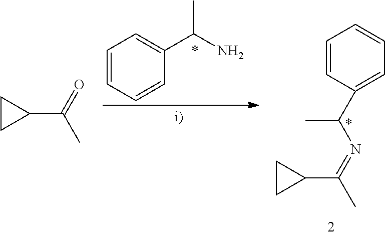

TABLE 1

| | | | | | | | | Conversion |
|---|---|---|---|---|---|---|---|---|
| | | Ketone | Amine | Cat. | B(OiPr)₃ | Temp | Time | a % (GC |
| Ent | Solvent | Eq. | Eq. | Eq. | Eq. | ° C. | h | Method 1) |
| 1 | THF | 1.5 | 1.0 | / | / | 60-80 | 10 | 13 |
| 2 | THF | 1.5 | 1.0 | 5% | / | 60-80 | 10 | 24 |
| | | | | TsOH · H₂O | | | | |
| 3 | MeOH | 1.0 | 1.0 | / | 1.1 | 60-80 | 10 | 8 |
| 4 | Toluene | 1.0 | 1.0 | / | 1.1 | 60-80 | 10 | 40 |
| 5 | IPAc | 1.0 | 1.0 | / | 1.1 | 60-80 | 10 | 33 |
| 6 | THF | 1.0 | 1.0 | / | 1.1 | 60-80 | 10 | 38 |
| 7 | IPA | 1.0 | 1.0 | / | 1.1 | 60-80 | 10 | 27 |

Solvents screening (Table 2):

Since the peak of amine was broad in GC, the results were calculated based on area % of imine/area % of ketone (GC Method 1).

TABLE 2

| | | Ketone | Amine | Cat. | B(OiPr)₃ | Temp. | Time | Conversion |
|---|---|---|---|---|---|---|---|---|
| Ent | Solvent | Eq. | Eq. | Eq. | Eq. | ° C. | h | % |
| 1 | Tol | 1.5 | 1.0 | 5% ZnCl2 | 1.2 | 60-80 | 10 | 39 |
| 2 | IPAc | 1.5 | 1.0 | 5% ZnCl2 | 1.2 | 60-80 | 10 | 41 |
| 3 | 2-MeTHF | 1.5 | 1.0 | 5% ZnCl2 | 1.2 | 60-80 | 10 | 45 |
| 4 | IPA | 1.5 | 1.0 | 5% ZnCl2 | 1.2 | 60-80 | 10 | 17 |
| 5 | heptane | 1.5 | 1.0 | 5% ZnCl2 | 1.2 | 60-80 | 10 | 50 |
| 6 | THF | 1.5 | 1.0 | 5% ZnCl2 | 1.2 | 60-80 | 10 | 60 |

Note:
Even though the equivalent of ketone was excess (1.5 eq), all the reactions were uncompleted and both SMs remained.

Formation of Mandelic Acid Salt of Formula IV (Step 2):

The solution of 4 (assay: 59.7 g) was charged with (R)-mandelic acid (106.7 g) and stirred for 1 h at room temperature. The mixture was concentrated to 350 mL, and EtOH (215 mL) was added. It was heated to reflux and MTBE (900 mL) was added slowly for 1 h. The mixture was stirred at reflux for 1 h, then cooled to 5° C. The precipitate was filtered and washed with a mixture of EtOH (30 mL) and MTBE (90 mL). The cake was dried and then recrystallized from MTBE/EtOH again to give 102.2 g compound 1 as white solid in 61% yield (GC Method 1), 99.8% ee.

GC-MS (Method 2): R₄=5.5 min

MS (Method 4): m/z=85.1 M⁺

¹H NMR (400 MHz, DMSO-d₆): δ 0.16-0.24 (m, 1H), 0.30-0.38 (m, 1H), 0.38-0.48 (m, 2H), 0.8-0.9 (m, 1H), 1.16 (d, 3H), 2.34-2.42 (m, 1H), 4.52 (s, 1H), 7.12-7.17 (m, 1H), 7.20-7.25 (m, 2H), 7.34-7.38 (m, 2H), 7.6-8.6 (br, 2H).

¹³C NMR (400 MHz, DMSO-d₆): δ 180.0, 148.9, 132.5, 131.5, 131.2, 78.7, 56.6, 23.6, 20.4, 9.0, 8.0

1. A process for preparing a non-racemic 1-cyclopropyl alkyl-1-amine of formula I 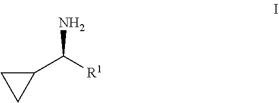
comprising the reaction of a compound of formula II 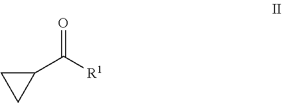
with a compound of formula III 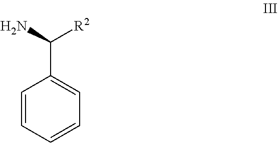
in which R¹ and R² are independently C₁-C₆-alkyl.

2. The process of claim 1, wherein the process comprises the steps of i) condensation of a compound of formula II with a compound of formula III to form an imine of formula INT1, ii) reduction to a secondary amine of formula INT2, and iii) debenzylation of the amine of formula INT2 to the compound of formula I

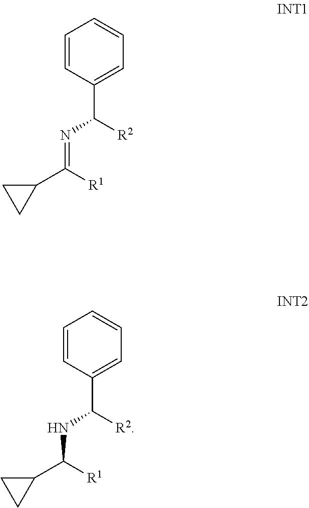

3. The process of claim 2, wherein a Lewis acid is used in step i).

4. The process of claim 3, wherein the Lewis acid is B(OiPr)₃ or Ti(OiPr)₄.

5. The process of claim 2, wherein step i) is run in a solvent selected from the group consisting of methanol, ethanol, iso-propanol, benzene, toluene, hexane, heptane, cyclopentane, cyclohexane, THF, and 2-MeTHF.

6. The process of claim 2, wherein step ii) comprises the use of NaBH₄ or LiBH₄.

7. The process of claim 2, wherein step ii) is run in a solvent selected from the group consisting of methanol, ethanol, iso-propanol, THF, and mixtures thereof.

8. The process of claim 2, wherein step iii) comprises the use of Pd as catalyst under hydrogen atmosphere.

9. The process of claim 8, wherein the Pd catalyst is Pd/C or Pd(OH)₂/C.

10. The process of claim 2, wherein step iii) is run in a solvent selected from the group consisting of methanol, ethanol, iso-propanol, and mixtures thereof.

11. The process of claim 1, wherein R¹ is C₁₋₃-alkyl.

12. The process of claim 1, wherein R² is methyl.

13. The process of claim 1, wherein the optical purity of 1-cyclopropyl alkyl-1-amines of formula I is 60% ee or higher.
